(19) United States
(12) Patent Application Publication
(10) Pub. No.: US 2019/0110990 A1
(43) Pub. Date: Apr. 18, 2019

(54) LIPOPROTEINS CONTAINING PLATINUM COMPLEXES FOR THE TREATMENT OF CANCER

(71) Applicants: UNIVERSITE DE BOURGOGNE, DIJON (FR); INSERM (INSTITUT NATIONAL DE LA SANTÉ ET DE LA RECHERCHE MÉDICALE), Paris (FR); CENTRE HOSPITALIER UNIVERSITAIRE, DlJON CEDEX (FR)

(72) Inventors: Frédéric LIRUSSI, COUTERNON (FR); Carmen GARRIDO-FLEURY, TALANT (FR); Laurent LAGROST, DIJON (FR)

(21) Appl. No.: 16/305,999

(22) PCT Filed: May 29, 2017
(86) PCT No.: PCT/FR2017/051320

§ 371 Date: Nov. 30, 2018

(30) Foreign Application Priority Data
May 31, 2016 (FR) 1654931

(51) Int. Cl. A61K 9/127; A61K 33/243; A61P 35/00

(52) U.S. Cl. CPC A61K 9/1275; A61P 35/00; A61K 33/243

(57) ABSTRACT

The present invention relates to lipoproteins containing platinum complex. The invention also relates to a kit comprising said lipoproteins. In particular, the present invention relates to the use of said platinum-complex-bearing lipoproteins for the specific targeting of macrophages and tumour cells in the treatment of cancer.

### FIELD OF THE INVENTION

The present invention relates to the use of anti-tumour agents for the treatment of cancer.

### INTRODUCTION

Platinum complexes are routinely used in cancer treatment. Among these, mention may be made of cisplatin, carboplatin, oxaliplatin, tetraplatin, iproplatin, satraplatin, nedaplatin, lobaplatin, picoplatin (Pt-based drugs: The spotlight will be on proteins, O. Pinato, C. Musetti and C. Sissi, Metallomocis February 2014) or ProLindac (ProLindac™ (AP5346): A review of the development of an HPMA DACH platinum Polymer Therapeutic, David P Nowotnika, Esteban Cvitkovic, Advanced Drug Delivery Reviews Volume 61, Issue 13, 12 November 2009, Pages 1214-1219). However, these platinum complexes as currently used have the major drawback of forming adducts with proteins of which albumin (Cisplatin Binding Sites on Human Albumin, Andrei I. Ivanov, John Christodoulou, John A. Parkinson, Kevin J. Barnham, Alan Tucker, John Woodrow, and Peter J. Sadler, The Journal of Biological Chemistry, Vol. 273, No. 24, Issue of June 12, pp. 14721-14730, 1998). Cisplatin is one of the most commonly used platinum complexes.

Cis-diamine-dichloro-platinum (II) (CDDP) complex, better known as cisplatin, is an antineoplastic used in cancer treatment. However, like most antineoplastics used in cancer therapies, cisplatin is not suitable for specifically targeting cancer cells. Moreover, the use thereof is accompanied by side-effects such as nephrotoxicity, neurotoxicity, ototoxicity, toxicity for bone marrow and other tissues, haemolysis, peripheral neuropathy and gastrointestinal irritation accompanied by nausea and vomiting.

It is therefore necessary to find methods for increasing the efficacy and selectivity of platinum complexes, in particular of cisplatin, while reducing the toxicity associated with the use thereof.

### SUMMARY OF THE INVENTION

The present invention relates to a low-density lipoprotein (LDL) charged with platinum complex. A further aim of the present invention relates to a high-density lipoprotein (HDL) charged with platinum complex or a modified low-density lipoprotein charged with platinum complex.

Furthermore, a further aim of the invention relates to a kit comprising:

a high-density lipoprotein (HDL) charged with platinum complex or a modified low-density lipoprotein charged with platinum complex or a mixture thereof, and

a low-density lipoprotein (LDL) charged with platinum complex.

The present invention also relates to a low-density lipoprotein (LDL) charged with platinum complex for use thereof against cancer, characterised in that it is used in combination with a modified low-density lipoprotein charged with platinum complex. Similarly, the present invention relates to a high-density lipoprotein (HDL) charged with platinum complex for use thereof against cancer, characterised in that it is used in combination with a low-density lipoprotein (LDL) charged with platinum complex.

The inventors of the present invention demonstrated that vectorisation of a platinum complex, in particular cisplatin, made it possible to increase the efficacy of said complex in cancer treatment, while helping reduce the toxicity associated with the use thereof. The inventors also demonstrated that the combination of different types of lipoproteins charged with platinum complex, in particular cisplatin, made it possible to obtain a synergistic effect and therefor further improve the efficacy of said complex in cancer treatment while reducing the toxicity thereof for the body.

### DETAILED DESCRIPTION

Low-density lipoproteins (LDLs) and high-density lipoproteins (HDLs) are well-known to those skilled in the art (Introduction to Lipids and Lipoproteins, Kenneth R Feingold MD, Car Grunfeld, PhD, NCBI Bookshelf).

Typically, high-density lipoproteins (HDLs) are lipoproteins rich in cholesterol, phospholipids and comprising apolipoproteins A-I, A-II, A-IV, C-I, C-II, C-III, and E, of density between 1.063 and 1.210 g/mL and of diameter varying between 5 and 12 nm.

Typically, native low-density lipoproteins (LDLs), in non-oxidised and non-acetylated form, are lipoproteins rich in cholesterol and comprising apolipoprotein B-100, of density between 1.019 and 1.063 g/mL and of diameter varying between 18 and 25 nm.

According to the present invention, the term “modified low-density lipoprotein” or “modified LDL lipoprotein” denotes an oxidised or acetylated low-density lipoprotein (LDL).

Typically, HDL and LDL lipoproteins are obtained from donors' plasma by means of an ultracentrifugation separation technique.

Typically, for the production of a lipoprotein (LDL, HDL or modified LDL) charged with platinum complex, the platinum complex is added to the lipoprotein in physiological medium. Typically, in order to enable the binding of said platinum complex with the lipoprotein and eliminate the unbound fraction of said platinum complex, the samples are incubated then dialysed.

Typically, the concentration of said platinum complex is determined by graphite furnace atomic absorption spectrometry.

Typically, the platinum complex concentration detected in the lipoproteins is from 0.1 to 1 mg/mL, preferentially from 0.2 to 0.8 mg/mL and even more preferentially from 0.3 to 0.6 mg/mL of final solution, i.e. the solution obtained by adding lipoproteins in a phosphate buffered saline (PBS) solution containing cis-platin. This concentration is measured for a cholesterol concentration of 1 mmol/mL of said final solution.

Typically, the platinum complex concentration detected in the LDL lipoproteins is 0.3 mg/mL of said final solution. This concentration is measured for a cholesterol concentration of 1 mmol/mL of said final solution.

Typically, the platinum complex concentration detected in the HDL lipoproteins is 0.5 mg/mL of said final solution. This concentration is measured for a cholesterol concentration of 1 mmol/mL of said final solution.

The present invention relates to a low-density lipoprotein (LDL) charged with platinum complex.

According to the present invention, said charged low-density lipoprotein (LDL) is used as a medicament.

More particularly, said charged low-density lipoprotein (LDL) is used to treat cancer.

Any type of cancer may be treated, in particular cancers for which platinum complexes are already routinely used: colorectal cancer, colon cancer, stomach cancer, ear, nose and throat (ENT) cancers, breast cancer, pancreatic cancer, liver cancer, lung cancer, brain cancer, prostate cancer, ovarian cancer, testicular cancer, oesophageal cancer, bladder cancer, epidermoid cancers, cervical cancer, endometrial cancer, bone cancer, lymphomas, central nervous system tumours, sarcomas, leukaemias and adenomas.

In one particular embodiment, the cancer is colorectal cancer or breast cancer.

Even more particularly, said charged low-density lipoprotein (LDL) is used in therapy to induce tumour cell death by apoptosis.

The present invention also relates to a high-density lipoprotein (HDL) charged with platinum complex or a modified low-density lipoprotein charged with platinum complex.

Modified low-density lipoproteins (LDLs) are known by those skilled in the art to be recognised by macrophage scavenger receptors. Typically, they may be obtained by incubation in the presence of copper sulphate or of a free radical generator (oxidised LDL) or by acetylation (acetylated LDL) (see A Modification Method for Isolation and Acetylation of Low Density Lipoprotein of Human Plasma by Density Discontinuous Gradient Ultracentrifugation, J. Z. Reza et al., Journal of Biological Sciences 10 (8): 785-789, 2010 ISSN 1727-3048).

According to the present invention said charged high-density lipoprotein (HDL) or said charged modified low-density lipoprotein is used as a medicament.

More particularly, said charged high-density lipoprotein (HDL) or said charged modified low-density lipoprotein is used to treat cancer.

Any type of cancer may be treated, in particular cancers for which platinum complexes are already routinely used: colorectal cancer, colon cancer, stomach cancer, ear, nose and throat (ENT) cancers, breast cancer, pancreatic cancer, liver cancer, lung cancer, brain cancer, prostate cancer, ovarian cancer, testicular cancer, oesophageal cancer, bladder cancer, epidermoid cancers, cervical cancer, endometrial cancer, bone cancer, lymphomas, central nervous system tumours, sarcomas, leukaemias and adenomas.

In one particular embodiment, the cancer is colorectal cancer or breast cancer.

More particularly, said charged high-density lipoprotein (HDL) or said charged modified low-density lipoprotein is used in therapy to activate macrophages.

Typically, the macrophage activation induces a production of numerous secretion products involved in inflammation. Secretion products involved in inflammation are for example reactive oxygen species (ROS), enzymes (proteases and lipases), cytokines or coagulation components.

The present invention also relates to a kit comprising:

a high-density lipoprotein (HDL) charged with platinum complex or a modified low-density lipoprotein charged with platinum complex or a mixture thereof, and

a low-density lipoprotein (LDL) charged with platinum complex.

More particularly the kit is used to treat cancer.

Any type of cancer may be treated, in particular cancers for which platinum complexes are already routinely used: colorectal cancer, colon cancer, stomach cancer, ear, nose and throat (ENT) cancers, breast cancer, pancreatic cancer, liver cancer, lung cancer, brain cancer, prostate cancer, ovarian cancer, testicular cancer, oesophageal cancer, bladder cancer, epidermoid cancers, cervical cancer, endometrial cancer, bone cancer, lymphomas, central nervous system tumours, sarcomas, leukaemias and adenomas.

In one particular embodiment, the cancer is colorectal cancer or breast cancer.

Furthermore, the present invention relates to a low-density lipoprotein (LDL) charged with platinum complex for use thereof against cancer, characterised in that it is used in combination with a modified low-density lipoprotein charged with platinum complex.

Similarly, the present invention relates to a high-density lipoprotein (HDL) charged with platinum complex for use thereof against cancer, characterised in that it is used in combination with a low-density lipoprotein (LDL) charged with platinum complex.

Any type of cancer may be treated, in particular cancers for which platinum complexes are already routinely used: colorectal cancer, colon cancer, stomach cancer, ear, nose and throat (ENT) cancers, breast cancer, pancreatic cancer, liver cancer, lung cancer, brain cancer, prostate cancer, ovarian cancer, testicular cancer, oesophageal cancer, bladder cancer, epidermoid cancers, cervical cancer, endometrial cancer, bone cancer, lymphomas, central nervous system tumours, sarcomas, leukaemias and adenomas.

In one particular embodiment, the cancer is colorectal cancer or breast cancer.

The present invention also relates to a pharmaceutical composition comprising a high-density lipoprotein (HDL) charged with platinum complex as defined above or a modified low-density lipoprotein charged with platinum complex as defined above.

The present invention also relates to a pharmaceutical composition comprising a low-density lipoprotein (LDL) charged with platinum complex as defined above.

The present invention also relates to the use of said lipoproteins charged with platinum complex as defined above or of said kit as defined above for producing a medicament intended for cancer treatment.

The present invention also relates to a method for the treatment of cancer which comprises the administration to a patient of a therapeutically effective quantity of said lipoproteins charged with platinum complex as defined above.

The term therapeutically effective quantity denotes any quantity of charged lipoproteins according to the present invention which is sufficient to induce an anti-tumour response or activate macrophages.

As mentioned above, platinum complexes are routinely used in cancer treatment. Examples of platinum complex are cisplatin, carboplatin, oxaliplatin, tetraplatin, iproplatin, satraplatin, nedaplatin, lobaplatin, picoplatin or ProLindac (polymer-platinate-DACH AP5346).

According to the present invention, “ProLindac” denotes a diaminocyclohexane (DACH)-platinum (Pt) complex coupled with hydroxypropylmethacrylamide (HPMA) copolymer (NCI Drug Dictionary, National Cancer Institute).

Thus, in one particular embodiment, the platinum complex is chosen from the group comprising cisplatin, carboplatin, oxaliplatin, tetraplatin, iproplatin, satraplatin, nedaplatin, lobaplatin, picoplatin and ProLindac (polymer-platinate-DACH AP5346).

In one preferred embodiment, the platinum complex is cisplatin.

### Summary of the Examples

The inventors demonstrated that vectorisation of platinum complexes, in particular cisplatin, was possible via high-density lipoproteins (HDLs) but also via low-density lipoproteins (LDLs).

In comparison with non-vectorised platinum complexes, vectorisation of platinum complexes, in particular cisplatin, by lipoproteins makes it possible to enhance the efficacy of the anti-tumour response of said complex while reducing the toxicity associated with the use of platinum complexes.

Lipoproteins charged with platinum complex, in particular cisplatin, as defined according to the present invention, make it possible to target different cell types effectively. Typically, charged HDL and modified LDL type lipoproteins make it possible to target macrophages and charged LDL type lipoproteins make it possible to target tumour cells. Furthermore, the vectorisation of platinum complexes, in particular cisplatin, by lipoproteins, makes it possible to activate macrophages further and target tumour cells further compared to non-vectorised platinum complexes.

More particularly, the use of both LDL lipoproteins charged with platinum complex and the use of HDL lipoproteins charged with platinum complex or of modified LDL lipoproteins charged with platinum complex or the mixture thereof, helps provide a synergistic effect and therefore enhance anti-tumour efficacy by targeting two cell types specifically and simultaneously. Indeed, the combined use of LDL lipoproteins charged with cisplatin and the use of HDL lipoproteins charged with platinum complex or of modified LDL lipoproteins charged with platinum complex or a mixture thereof, makes it possible to target specifically both tumour cells and macrophages. This use therefore makes it possible to apply a more powerful cytotoxic effect and strengthen the immune response via macrophage activation while reducing the toxicity associated with the use of the platinum complex alone.

### FIGURES

FIG. 1: Study of cisplatin vectorisation in LDLs and HDLs

FIG. 1A: Cisplatin vectorisation

FIG. 1B: Evaluation of cisplatin exchanges between charged LDLs/HDLs and native LDLs/HDLs

FIG. 2: Effect of cisplatin vectorisation on cancer cells and on macrophages

FIG. 2A: Effect of cisplatin vectorisation on tumour cells

FIG. 2B: Effect of cisplatin vectorisation on macrophages

FIG. 2C: Effect of cisplatin vectorisation on macrophages (with oxidised LDL)

FIG. 3: Study of potency of LDLs charged with cisplatin and HDLs charged with cisplatin on cancer cells and on macrophages in tumour extracts

FIG. 4: Cisplatin vectorisation by LDLs—enhancement of tumour efficacy—in vivo

FIG. 4A: Progression of tumour size over time

FIG. 5: Cisplatin vectorisation by LDLs—reduction of toxicity in vivo

FIG. 5A: Effect of cisplatin vectorisation by LDLs—tumour volume

FIG. 5B: Effect of cisplatin vectorisation by LDLs—weight loss

### EXAMPLES

### Preparation of Lipoproteins Charged with Cisplatin

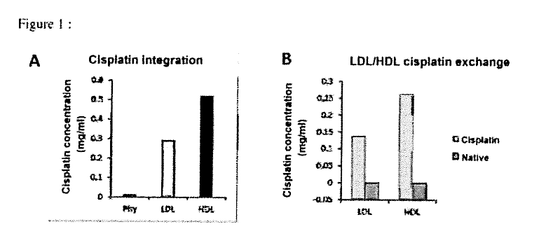

Low-density lipoproteins and high-density lipoproteins were isolated from plasma from healthy donors by a potassium bromide (KBr)-differential density gradient ultracentrifugation separation technique (Redgrave technique, 1975). After extraction, the lipoproteins were adjusted to a cholesterol concentration of 1 mM. 100 μl of a cisplatin solution (at 10 mg/ml, in physiological saline solution) was then added for an expected final concentration of 1 mg/ml. In order to enable the binding of cisplatin with the lipoproteins and eliminate the unbound fraction of cisplatin, the samples were incubated for 3 hours at 37° C., then subjected to two successive dialyses (against 1000 times the volume of phosphate buffered saline (PBS), Cutoff 7000 Da) of 1 hour and 18 hours respectively. After dialysis, the cisplatin concentration was determined by graphite furnace absorption spectrometry (GF-AAS) (FIG. 1A). As demonstrated in FIG. 1A, the cisplatin concentration in the LDLs is 0.3 mg/mL of final solution, i.e. the solution obtained by adding lipoproteins in a phosphate buffered saline (PBS) solution containing cisplatin. The cisplatin concentration in the HDLs is 0.5 mg/mL of said final solution. These concentrations are measured for a cholesterol concentration of 1 mmol/mL of said final solution.

Thus, over 30% of the initial cisplatin concentration was vectorised in purified HDL and LDL fractions.

### Stability study of Lipoproteins Charged with Cisplatin (FIG. 1B)

LDLs containing vectorised cisplatin (LDL-Cis) were incubated for 18 hours at 37° C. with native HDLs (HDL 0). Similarly, HDLs containing vectorised cisplatin (HDL-Cis) were therefore incubated for 18 hours at 37° C. with native LDLs (LDL 0).

After incubation, these lipoprotein fractions were extracted with a potassium bromide (KBr)-differential density gradient ultracentrifugation separation technique. The quantity of cisplatin bound to the different fractions was then determined by graphite furnace absorption spectrometry (GF-AAS).

As demonstrated in FIG. 1B, after 18 hours of incubation, 0.13 mg and 0.26 mg of cisplatin per mL of said final solution were still present respectively in the LDL-Cis and HDL-Cis fractions. On the other hand, no trace of cisplatin was detected in the native LDL and HDL fractions (FIG. 1B).

These results demonstrate therefore that binding of cisplatin with the lipoproteins is stable. Indeed, after 18 hours of incubation, approximately 50% of the initially vectorised cisplatin was still present in the lipoprotein fractions, and no exchange of cisplatin with other lipoprotein classes occurred.

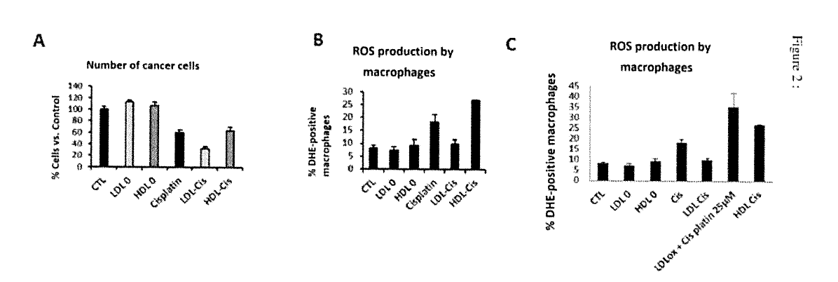

### In Vitro Study of Effects of Cisplatin Vectorisation by HDL and LDL Lipoproteins on Adenocarcinoma Cells or Macrophages in Culture (FIG. 2A)

Adenocarcinoma cells and macrophages are mostly detected in colon tumours.

For this test, SW480 colorectal cancer lines were treated for 48 hours with native LDLs (LDL 0), native HDLs (HDL 0), non-vectorised cisplatin, LDL-Cis or HDL-Cis (final cisplatin concentration: 25 μM). The cell viability was then evaluated by flow cytometry. As per FIG. 2A, the non-vectorised cisplatin induced a 41% cancer cell mortality. On the other hand, the native LDLs and HDLs did not induce any effect on the cancer cells. Moreover, the HDL-Cis induced a 37% cancer cell mortality, which is comparable to the effect of non-vectorised cisplatin. On the other hand, the LDL-Cis induced a 58% mortality of the SW480 cells, i.e. a much superior effect to that obtained for non-vectorised cisplatin (FIG. 2A).

### Study of the Impact of Cisplatin Vectorisation on ROS Production (FIG. 2B)

After 7 days of culture in human M-CSF from Miltenyi, Biotec. (Macrophage Colony-Stimulating Factor) at 100 ng/ml, human macrophages were differentiated, from monocytes, into M2 alternative phenotype macrophages (protumoral). These macrophages were then stimulated for 2 hours with native LDLs (LDL 0), native HDLs (HDL 0), non-vectorised cisplatin, LDL-Cis or HDL-Cis (final cisplatin concentration: 25 μM). The production of reactive oxygen species (ROS, representative of an anti-tumour action) by the macrophages was then determined by flow cytometry after labelling with Dihydroethidium (DHE). This test demonstrates therefore that the use of non-vectorised cisplatin makes it possible to increase the basal ROS production from 8.2% to 18.2% by macrophages compared with the control sample (CTL). Furthermore, the native HDLs, native LDLs and the LDL-Cis had no effect on ROS production by the macrophages. On the other hand, the HDL-Cis induce 26.8% macrophage activation, i.e. an approximately 50% more effective effect than non-vectorised cisplatin (FIG. 2B).

### Study of the Impact of Cisplatin Vectorisation on ROS Production for Oxidised LDLs Charged with Cisplatin (FIG. 2C)

The same protocol was repeated so as to compare the effect between the HDL-Cis and the oxidised LDL lipoproteins charged with cisplatin (LDLox+Cis) (see FIG. 2C). Thus, the LDLox+Cis induce superior macrophage activation to that induced by the HDL-Cis. The oxidised LDLs were obtained by incubating native

LDLs (cholesterol, 1 mM) for 24 hours at 37° C. in the presence of copper sulphate (5 μM). After oxidation, the oxidised LDLs are dialysed in a PBS buffer.

### Specific Targeting

The above tests make it possible therefore to demonstrate that, in vitro, charged LDLs appear to have an effect only on cancer cells, whereas charged HDLs have an effect only on macrophages. The vectorisation of cisplatin by HDLs makes it possible to increase the efficacy of the treatment by almost 50% compared to non-vectorised cisplatin. Similarly, cisplatin vectorisation by LDLs makes it possible to increase the efficacy of the treatment by almost 50% compared to non-vectorised cisplatin.

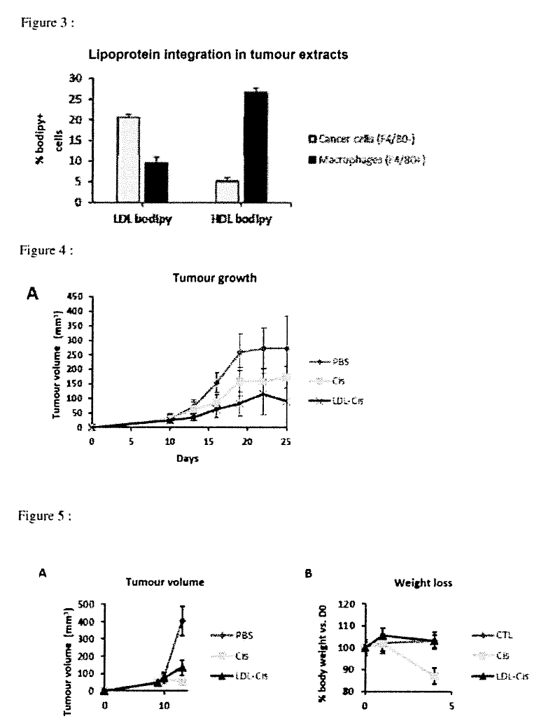

In a further test, tumours obtained from an ectopic (subcutaneous) allograft model and CT-26 colon tumours in BALB-C mice were isolated and placed in contact with LDL or HDL type fluorescent lipoproteins (bodipy) (FIG. 3). As shown in FIG. 3, the LDLs are preferentially taken up by the tumour cells whereas HDLs are for their part mostly taken up by macrophages.

### In Vivo Tests

In order to verify the in vitro and ex vivo results above for an in vivo model, the ectopic (subcutaneous) allograft model of CT-26 colon tumours in BALB-C mice was used. As shown by FIG. 4A, after 25 days of treatment, the mice treated by 1.5 mg/kg LDL-Cis exhibit much less developed tumours compared with the control group (CLT) and with the non-vectorised 1.5. mg/kg cisplatin group. Furthermore, as proven by the histological tumour analysis (not shown), vectorisation enhances the production of radical species (DHE) and induces more apoptosis (caspase-3 cleavage) compared to the non-vectorised cisplatin group. These experiments therefore demonstrated that the vectorisation of a cytotoxic agent enhanced the anti-tumour efficacy thereof.

### Study of Nephrotoxicity and Other Side-Effects for Vectorised Cisplatin

The purpose of this test is to verify that the vectorisation of cisplatin indeed makes it possible to reduce systemic and renal toxicity compared to non-vectorised cisplatin. For this test, cisplatin was administered at a dose of 20 mg/kg for 3 days to our mouse model described above, i.e. the ectopic (subcutaneous) allograft model of CT-26 colon tumours in BALB-C mice (validated cisplatin-induced nephrotoxicity protocol).

As demonstrated in FIG. 5B, non-vectorised cisplatin induces, on one hand, weight loss of almost 15% compared to the control sample (CTL). Moreover, as proven by the histological analyses (not shown), non-vectorised cisplatin induces a high level of nephrotoxicity which is characterised by deepithelialisations, the presence of hyaline bodies and necrosis and apoptosis phenomena. In comparison, no weight loss or signs of nephrotoxicity were observed for the LDL-vectorised cisplatin group (see FIG. 5B). Thus, the vectorisation of cisplatin makes it possible to reduce the toxicity associated with the use thereof compared to non-vectorised cisplatin. Moreover, cisplatin vectorised by LDLs induces apoptosis of the cells within the tumour but not that of renal cells (histological analysis not shown). The use of vectorised cisplatin therefore makes it possible to do away with the side-effects associated with the use of non-vectorised cisplatin.

1. A lipoprotein wherein
the lipoprotein is a low-density lipoprotein (LDL) charged with platinum complex, or
the lipoprotein is a high-density lipoprotein (HDL) charged with platinum complex, or
the lipoprotein is an oxidised low-density lipoprotein charged with platinum complex, or
the lipoprotein is an acetylated low-density lipoprotein charged with platinum complex.

2-8. (canceled)

9. Kit wherein the kit comprises:
a high-density lipoprotein (HDL) charged with platinum complex or an oxidised or acetylated low-density lipoprotein charged with platinum complex or a mixture thereof, and
a low-density lipoprotein (LDL) charged with platinum complex.

10-12. (canceled)

13. Lipoprotein according to claim 1, wherein the platinum complex is selected from the group consisting of cisplatin, carboplatin, oxaliplatin, tetraplatin, iproplatin, satraplatin, nedaplatin, lobaplatin, picoplatin and ProLindac (polymer-platinate-DACH AP5346).

14. (canceled)

15. Lipoprotein according to claim 1, wherein the platinum complex is cisplatin.

16. Kit according to claim 9, wherein the platinum complex is selected from the group consisting of cisplatin, carboplatin, oxaliplatin, tetraplatin, iproplatin, satraplatin, nedaplatin, lobaplatin, picoplatin and ProLindac (polymer-platinate-DACH AP5346).

17. Kit according to claim 9, wherein the platinum complex is cisplatin.

18. Method for the treatment of a disease, wherein said method comprises the administration to a patient of a therapeutically effective quantity of the lipoprotein according to claim 1.

19. Method according to claim 18, wherein the disease is cancer.

20. Method according to claim 18, wherein the low-density lipoprotein (LDL) charged with platinum complex induces tumour cell death by apoptosis.

21. Method according to claim 18, wherein the low-density lipoprotein (LDL) charged with platinum complex is used in combination with an oxidised or acetylated low-density lipoprotein charged with platinum complex.

22. Method according to claim 18, wherein:
the high-density lipoprotein (HDL) charged with platinum complex activates macrophages, or
the oxidised low-density lipoprotein charged with platinum complex activates macrophages, or
the acetylated low-density lipoprotein charged with platinum complex activates macrophages.

23. Method according to claim 18, wherein the high-density lipoprotein (HDL) charged with platinum complex is used in combination with a low-density lipoprotein (LDL) charged with platinum complex.

24. Method for the treatment of cancer which comprises the step of using the kit according to claim 9.
